(19) United States
(12) Patent Application Publication
(10) Pub. No.: US 2015/0305923 A1
(43) Pub. Date: Oct. 29, 2015

(54) HEATED MASSAGE STONE

(71) Applicant: Jeff Ebel, Surprise, AZ (US)

(72) Inventor: Jeff Ebel, Surprise, AZ (US)

(21) Appl. No.: 14/699,074

(22) Filed: Apr. 29, 2015

Related U.S. Application Data
(60) Provisional application No. 61/985,977, filed on Apr. 29, 2014.

(51) Int. Cl. A61F 7/00

(52) U.S. Cl. CPC A61F 7/00; A61F 2007/0088

(57) ABSTRACT

A heated massage stone having a first ceramic portion having a cavity within and an outer curved surface extending to a first portion inward angled surface that extends to a first ceramic portion ledge perimeter extending away from the first portion inward angled surface at a substantially perpendicular angle. A silicon gasket is attached to the first ceramic portion ledge perimeter, the silicon gasket having a first gasket segment attached substantially along the inward angled surface, and a second gasket segment attached and extending along the first ceramic portion ledge perimeter. A second ceramic portion also having a cavity within and having an outer curved surface extends to a second ceramic portion inward angled surface. The second ceramic portion inward angled surface has a second ceramic portion edge perimeter that is larger than the first ceramic portion ledge perimeter. The first ceramic portion and second ceramic portion are pressure fit together and sealed by the silicon gasket.

### BACKGROUND OF THE INVENTION

### Field of the Invention

The present invention relates to massage stones and in particular, heated massage stones.

### BRIEF DESCRIPTION OF THE DRAWINGS

FIG. 1a illustrates a perspective view of an embodiment of a massage stone according to the invention;

FIG. 1b shows a perspective view of an embodiment of a massage stone with the two ceramic portions separated;

FIG. 2a-2d illustrates a preferred silicon gasket 30;

FIG. 3 illustrates an exploded view of an embodiment;

FIG. 4 illustrates an embodiment of a circuit to power the heating element 30;

FIG. 5 illustrates the embodiment of invention and showing the heating element 30 adhered to the ceramic stone inner cavity and a thermistor 70 coupled to the heating element 30; and

FIGS. 6a-6d illustrate components for another embodiment of the massage stone that enables remote control of a plurality of massage stones from a remote control device.

### SUMMARY OF THE INVENTION

In general the heated massage stone of the invention includes a first ceramic portion having a cavity within and an outer curved surface extending to a first portion inward angled surface that extends to a first ceramic portion ledge perimeter extending away from the first portion inward angled surface at a substantially perpendicular angle. A silicon gasket is attached to the first ceramic portion ledge perimeter, the silicon gasket having a first gasket segment attached substantially along the inward angled surface, and a second gasket segment attached and extending along the first ceramic portion ledge perimeter. A second ceramic portion also having a cavity within and having an outer curved surface extends to a second ceramic portion inward angled surface. The second ceramic portion inward angled surface has a second ceramic portion edge perimeter that is larger than the first ceramic portion ledge perimeter. The first ceramic portion and second ceramic portion are pressure fit together and sealed by the silicon gasket.

An additional aspect includes that the second ceramic portion has a second ceramic portion inner surface and the massage stone further comprises a sealed heater. The sealed heater includes an adhesive layer coupled to the interior first half surface and a substantially flat electrically conductive and resistive element embedded within a non-conductive material. A printed circuit board having at least one heater conductor is coupled to the substantially flat heater and a charging port having a charging input coupled to the a surface selected from at least one of the first or second ceramic portion outer curved surfaces. A first switch is connected in a circuit comprising the substantially flat resistive element, the battery, and at least one battery lead coupled to a battery.

### DESCRIPTION OF THE EMBODIMENTS

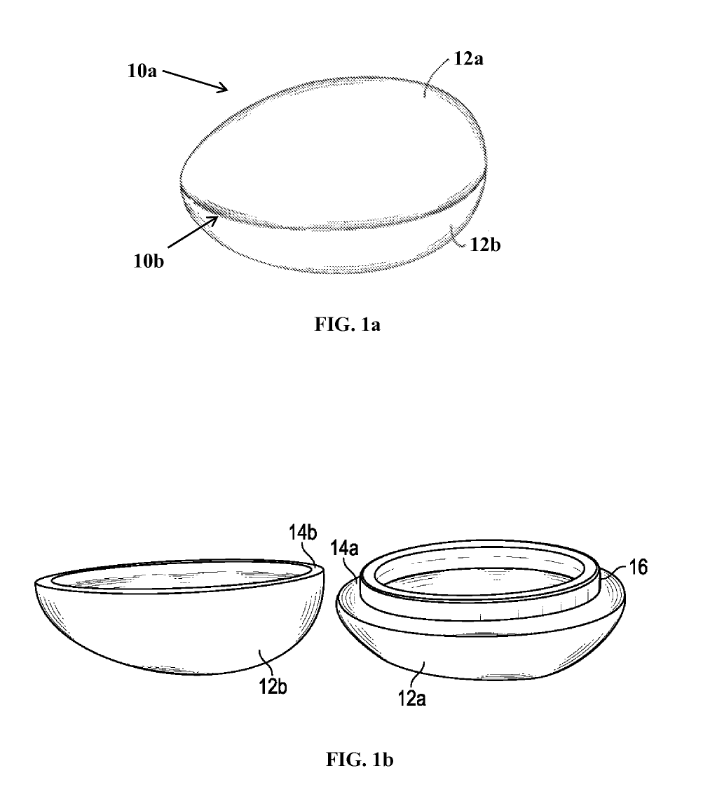

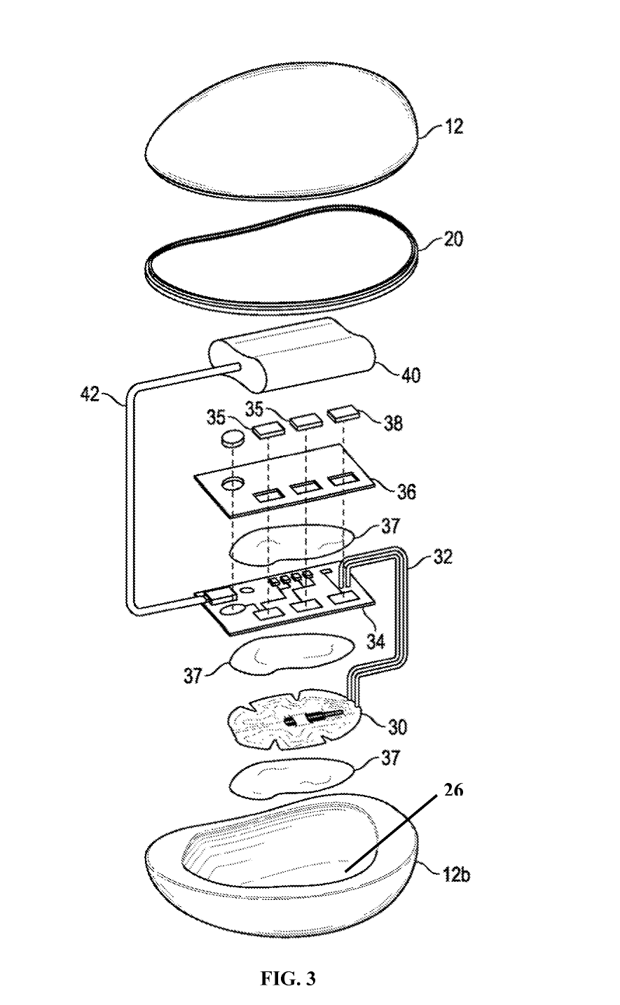

The heated massage stone comprises first ceramic portion and second ceramic portions that when attached together have a curved outer surface resembling a smooth surfaced stone such as common in tumbled or shaped and polished stones. See FIG. 1. As illustrated in FIG. 3, the heated massage stone seals and conceals a battery powered heater assembly. The first ceramic portion 10a and second ceramic portion 10b that connect together by a pressure fit. Moreover, while the description herein distinguishes between the first ceramic portion 10a and second ceramic portion 10b, it is within the knowledge of one of ordinary skill that the first and second ceramic portions, 10a and 10b, can be switched without departing from the scope or spirit of the invention and description herein.

The first ceramic portion 10a comprises an outer curved surface 12a that extends to a first ceramic portion negative edge or inward angled surface 14a. The inward angled surface 14a extends to a first ceramic portion ledge perimeter 16. See FIG. 1b. The first ceramic portion ledge perimeter 16 also comprises the edge and access to a cavity within the first ceramic portion 10a. The second ceramic portion 10b also has a second ceramic portion outer curved surface 12b and a second ceramic portion inward angled surface 14b constructed to match and pressure fit with the first ceramic portion outer curved surface 12a at the first ceramic portion inward angled surface 14a and second ceramic portion inward angled surface 14b, respectively.

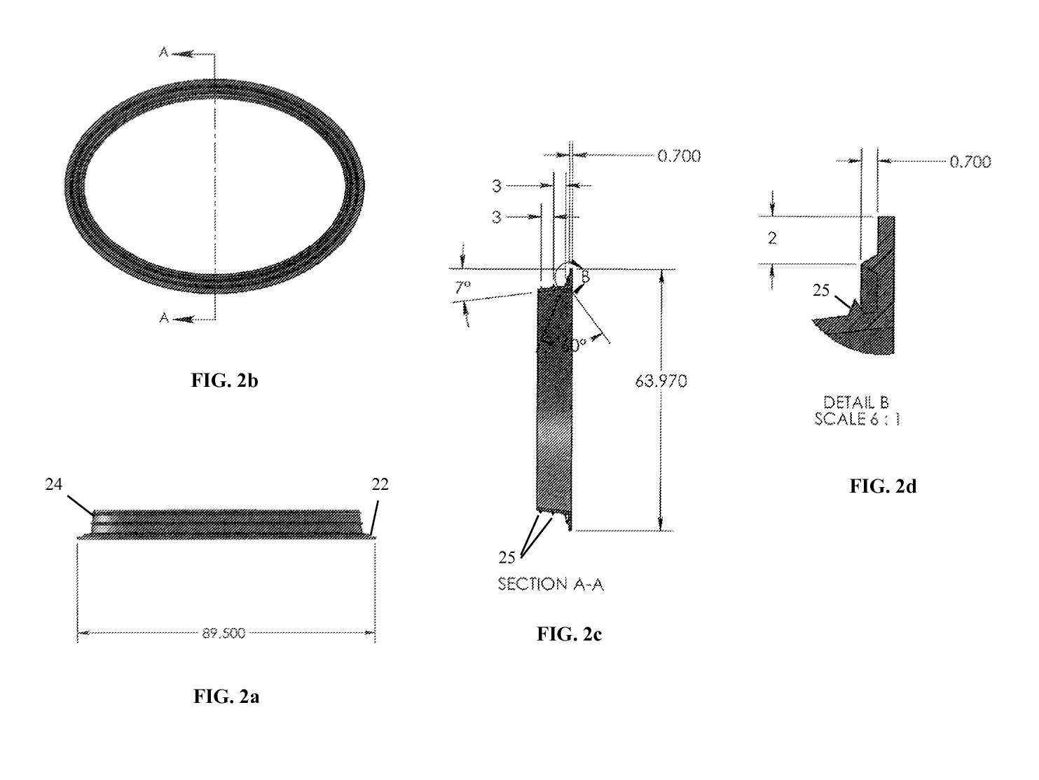

The pressure fit between the ceramic portions, 10a and 10b, is assisted by at least one silicon gasket 20 that attaches to the first ceramic portion 10a. In preferred embodiments, the silicon gasket 20 is attachable or firmly attached to both the first ceramic portion inward angled surface 14a and the first ceramic portion ledge perimeter 16. A preferred silicon gasket 20 is illustrated in FIGS. 2a-2d. A first gasket segment 22 attachable to and covering the length and width of the first ceramic portion negative edge or inward angled surface 14a and at least a second segment 24 attachable to and covering the length and width of the first ceramic portion ledge perimeter 16. See FIG. 2a. And, as illustrated in greater detail, see FIG. 2d, the preferred first gasket segment 22 comprises a first and a second thicker portion wherein the second thicker portion is about twice as thick as the first portion. Additionally, the second segment 24 preferably angles or curves away from the transition from the first segment 22 to the second segment 24 at an angle of between 91 and 105 degrees relative to the first ceramic portion inward angled surface 14a or between 1 and 15 degrees relative to a geometrical normal from the first ceramic portion inward angled surface 14a. See FIG. 2c. Moreover, as shown in FIG. 2d, the pressure fit is assisted by at least one protrusion 25, but preferably a plurality of protrusions 25, of and on, or from, the second segment 24 of the silicon gasket 30 that are selected from rectangular, curved, triangular, or cone-shaped and extending substantially perpendicularly from the second segment 24 and substantially parallel to the first segment 22. The protrusion 25 pivots or rocks slightly when contacted by the second ceramic portion inward angled surface 14b during a pressure fit sequence. FIGS. 2a and 2c, illustrate the preferred positioning of protrusions 25 being distributed evenly across the length of the second gasket segment 24. Finally, the drawing indicates preferred dimensions of the gasket 20 given in inches.

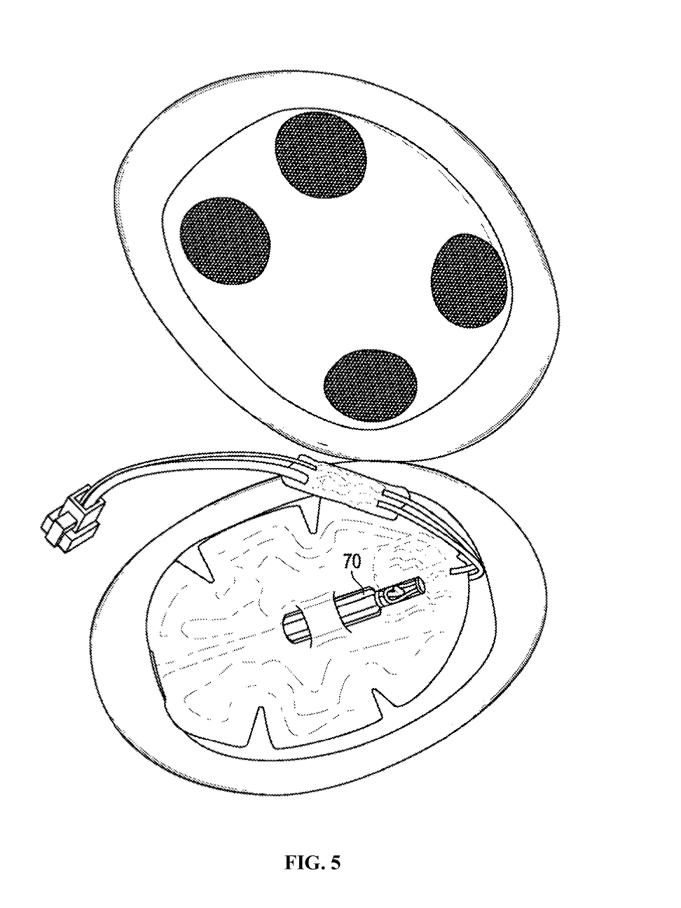

A heating element 30 comprised of a substantially flat electrically conductive and resistive element embedded within a non-conductive material is adhered to a second ceramic portion inner surface 26. See FIGS. 3 and 5. Heater leads 32 electrically couple the heating element 30 and the printed circuit board 34 and battery leads 42 electrically couple the printed circuit board 32 and the battery 40. A mask 36 is coupled to the printed circuit board on the side of the printed circuit board components 35. A charging port 38 having a charging input is coupled to a surface selected from at least one of the first or second ceramic portion outer curved surfaces 10a and 10b or at an intersection of the first or second ceramic portion outer curved surfaces 10a and 10b. Adhesive 37 is optionally included between the components comprising the sealed heater and preferentially between the printed circuit board 32 and the mask 36 and the heating element 30 and the second ceramic portion inner surface 26 and between the printed circuit board 34 and the heating element 30.

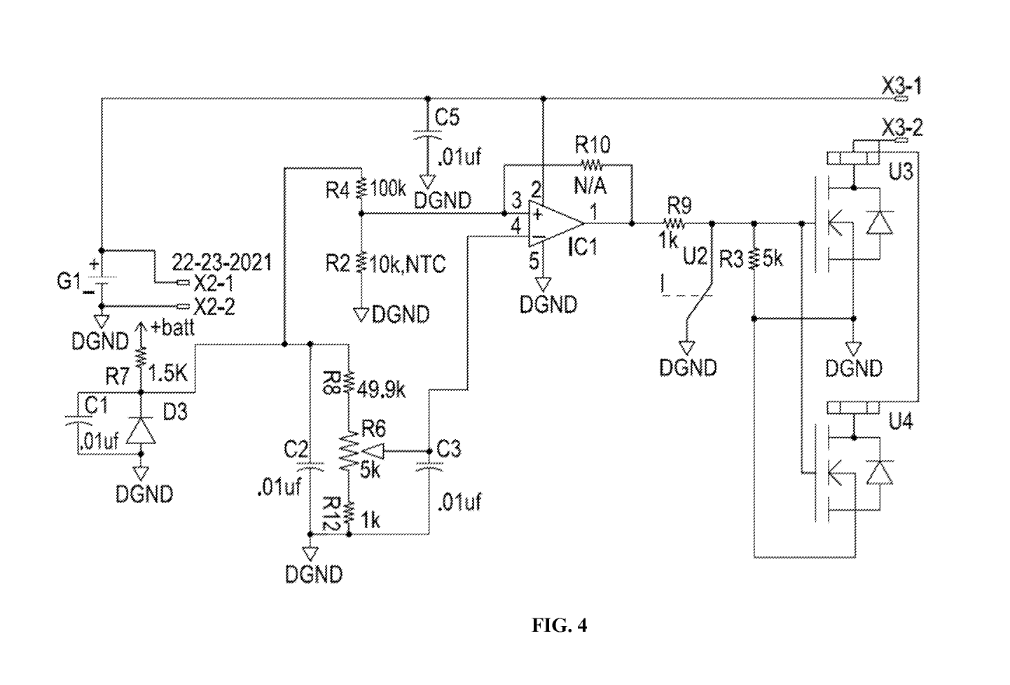

The printed circuit board 32 includes a circuit comprising a heating element 30 driver circuit to permit adjustable and pre-set temperature ranges. See FIG. 4. In general, a battery 40 is coupled to a switch U2 that electrically couples the battery 40 and the substantially flat electrically conductive and resistive element. A preferred circuit for powering the heating element 30 is illustrated in FIG. 3. The battery 40 is connected at G1 and a switch U2 is connected to the gates of FETs U3 and U4. The heating element 30 is connected between the battery and the drains of FETs U3 and U4 (i.e. between X3-1 and X3-2). When the switch U2 is permitting electrical current to flow, the transistors are “off” and prevent current flow through the drains of U3 and U4, which are in series and in the return path for current that would flow through the heating element 30. When the switch U2 is in position preventing current flow, the gates of FETs U3 and U4 are biased to a voltage based on the current flowing through resistor R5. The voltage at the gates of FETs U3 and U4 turns the FETs “on” to permit current flow from the battery 40, through the heating element 30, and through the drains and sources of FETs U3 and U4, to return to the battery 40. Potentiometer R6 provides adjustment of the voltage at the inverted input of the amplifier IC1 which sets the current through R5 and the biasing voltage at the gates of FETs U3 and U4. A greater differential voltage at the terminals 3 and 4 of IC1 provides greater current flow through R5 and a higher biasing voltage of FETs U3 and U4 and greater current through FETs U3 and U4 and a correspondingly higher temperature. A lower differential voltage at the terminals 3 and 4 of IC 1 provides reduced current flow through R5 and a lower biasing voltage for FETs U3 and U4 and less current through the FETs and a correspondingly lower temperature.

Finally, the thermistor 70 is a Negative Temperature Coefficient (NTC) thermistor which presents less resistance to current as its temperature increases, such as due to heating caused by the heating element 30, and is used to regulate the heating element 30 current. As the thermistor 70 heats the voltage at the non-inverting IC1 input will decrease and also decrease the bias voltage applied to the gates of FETs U3 and U4 and decrease the current flow through the heating element 30 thereby causing the heating element 30 and massage stone to cool. Conversely, as the thermistor 70 cools the voltage at the non-inverting IC1 input will increase and thereby increase the bias voltage applied to the gates of FETs U3 and U4 causing more current to flow through the heating element 30 thereby heating the massage stone. The adjustment of the potentiometer R6 sets the static operating temperature. In other embodiments a preset, rather than adjustable, heating element 30 temperature is provided to heat the massage stone. The heating element 30 is coupled into contact with second ceramic portion inner surface 26 with an optional adhesive or simply tacky material, which contact conducts heat from the heating element 30 to the second ceramic portion 10b.

FIGS. 5 illustrates an embodiment showing aspects of the preferred heating element 30 of the massage stone. The illustrated heating element 30 comprises a length of curved or undulating electrically-conductive material having electrically-resistive characteristics, which is positioned between layers of silicone insulating material. The preferred undulating electrically-conductive material having electrically-resistive characteristics comprises a graphite foil thin-film heater sandwiched between top and bottom silicone sheets, such as is available from EGC Enterprises, Inc. of Chardon Ohio 44024. Moreover as pictured, the preferred heating element 30 comprises a plurality or at least two loops of curved or undulating electrically-conductive material sandwiched between substantially flat and ovular shaped silicon sheets that are positioned in contact with the inner concave surface of the bottom stone half 12b. It is preferable that the electrically-conductive graphite foil traverse as much of the surface area of the silicon sheets as possible so as to maximize the transfer of heat from the electrically-conductive material through the silicon sheet to the ceramic stone inner cavity surface with which it contacts.

Assembly of the remaining components of the illustrated embodiment completes the assembly. A first graphite layer 80 having a thermistor shaped window 81 is placed over the heating element silicon layers and has a perimeter sized to match the diameter of the inner cavity of the second ceramic portion 10b. The thermistor shaped window 81 is positioned over the thermistor 80. A second graphite layer 80 also having a thermistor shaped window 81 is layered onto the first graphite layer 80. A battery 40 is placed in the cavity of the first ceramic portion 10a. Finally, a fire-retardant fabric sheet 90 with a perimeter sized to match the diameter of the inner cavity of the second ceramic portion 10b is placed over the second graphite layer 80. Finally, while the first and second ceramic portions 10a and 10b may be held together with magnetic connectors on the perimeter edge to hold the stone-halves together, the preferred manner of holding the portions 10a and 10b together comprises the pressure fit with gasket 20 described in FIGS. 2a-2d.

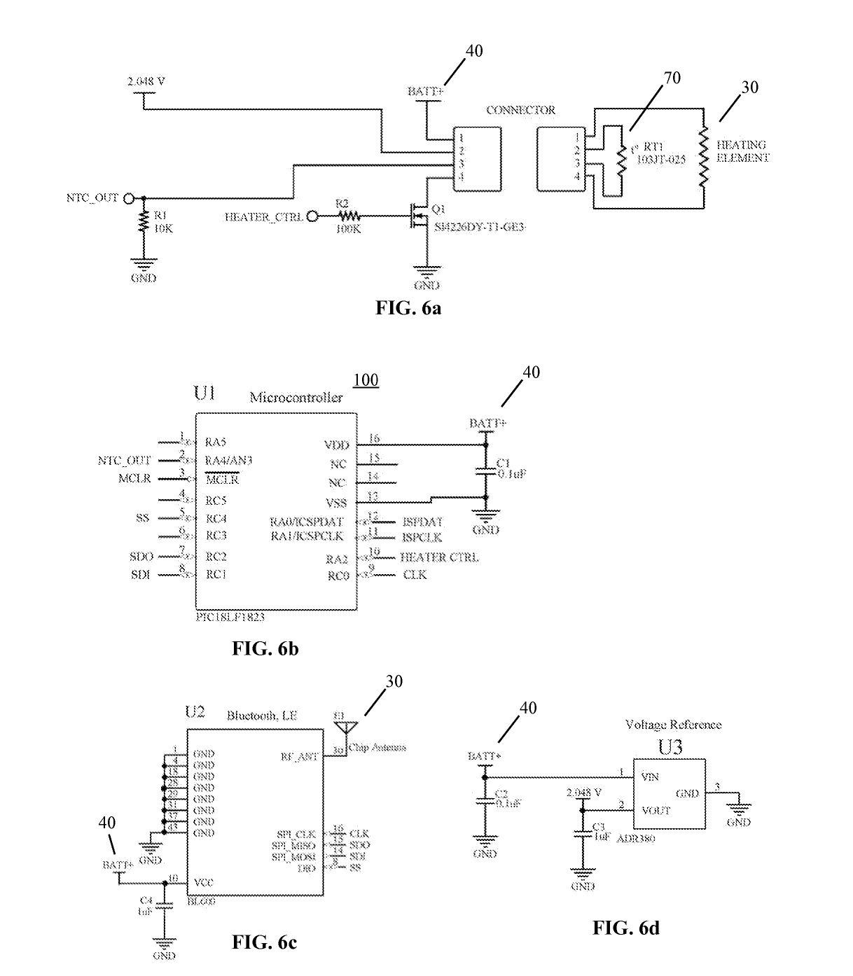

FIGS. 6a-6d illustrate components for an alternate embodiment that enables temperature control of a plurality of massage stone temperatures using a remote control device such as a user's computing device, smart phone, or other wireless remote or device. The remote control device is equipped with hardware and/or software to communicate wirelessly with each massage stone to set and/or modify control the heater circuit to adjust the massage stone temperature. The illustrated embodiment includes a wireless link, a microcontroller, a heater driver circuit, and a voltage reference circuit.

The preferred wireless link comprises a radio frequency wireless transceiver device, such as a Bluetooth® Low Energy device as illustrated in the drawings. Other low-energy wireless technologies, ANT, ANT+, ZigBee, ZigBee RF4CE, Wi-Fi, Nike+, IrDA are also acceptable technologies if used according to the teachings herein. The wireless link couples to the at least one remote control device to receive commands to control the heater driver circuit and adjust the massage stone temperature. Moreover, identification or distinction and control of any particular massage stone from a plurality of massage stones is enabled based on the design herein. Particularly, each massage stone will have a unique address or identification code so as to identify communications and commands intended for its control and thereby discriminate wireless communications and commands intended for control of another massage stone. For example, the use of a low energy Bluetooth® transceiver such as the B1600 Integrated Circuit to identify directed communications and discriminate from communications intended for other massage stones comprises use of the unique Bluetooth® MAC address in communications from the remote device to the massage stone.

Remote command and control of a particular massage stone by a remote device comprises transmission of a MAC address and accompanying commands to adjust or control a massage stone temperature. Conversely, control and command by a remote device comprises receipt of a MAC address and accompanying commands to adjust or control the massage stone temperature. By way of further explanation, each massage stone may be paired and identified with at least one remote control device and a remote control device may identify and be paired with one or a plurality of massage stones using the MAC addresses to distinguish between individual stones.

FIG. 6a illustrates another circuit to control the massage stone temperature and enables remote control of a plurality of massage stone temperatures from a single remote control device. The heating element 30 and the thermistor 70 are connected by leads through a connector to the PCB 34. On the PCB 34 the heating element 30 is coupled electrically in series (i.e. between terminals “1” and “4”) with the battery (“BATT+”) 40 and the drain of the n-channel mosfet “Q1”, the source of which is connected to ground. A resistor “R2” is coupled electrically in series between the gate of Q1 and a port (e.g. “RA2”) on a microcontroller 100. The microcontroller 100 is programmed to output a drive signal at RA2 to bias Q1 and enable current flow from the battery 40 through the heating element 30 through Q1 to ground, which current flow from the battery 40 dissipates from the heating element 30 to heat the ceramic portion inner surface 26. An NTC thermistor 70 (“RTI”), such as a 103JT-025, is mechanically coupled adjacent the heating element 30 and is coupled electrically in series (i.e. between terminals “2” and “3”) with a resistor “R1” between a static reference voltage (“2.048”) and ground. FIG. 6d illustrates an ADR380 Voltage reference device that creates the static reference voltage.

The node (“NTC_OUT”) between RT1 and R1 is coupled to an analog-to-digital (A/D) input port (“RA4/AN3”) of the microcontroller 100 that samples the voltage at NTC_OUT for operations within the microcontroller 100 as controlled by programming stored in memory in the microcontroller 100. The voltage and change in voltage NTC_OUT sampled by the RA4/AN3 input port will be proportional to the temperature or increase in temperature of the massage stone since the thermistor 70 is an NTC type. If the massage stone and heating element 30 temperature increases, the voltage at NTC_OUT will also increase since the resistance value of thermistor 70 has increased. Conversely, if the massage stone and heating element 30 temperature decreases, the voltage at NTC_OUT will also decrease since the resistance value of thermistor 70 has decreased. The microcontroller 100 has a program stored in memory to set and regulate the massage stone temperature by cycling Q1 on or off. The microcontroller 100 program calculates a massage stone temperature using the voltage sampled at NTC_OUT and the Steinhart-Hart equation and cycles Q1 on or off to achieve the target temperature.

The Bluetooth® BL600 wireless transceiver referenced in FIG. 6c includes a chip antenna “E1” 30 that is mounted to the PCB 34. The microcontroller 100 and BL600 are coupled together via the SPI buss to relay communications between the remote control device and the massage stone. In operation, a remote control device can transmit commands to turn “up” the heat, turn “down” the heat, or turn the unit off and the microcontroller 100 will process the commands from the remote control device while monitoring the temperature using the voltage sampled at NTC_OUT. The microcontroller 100 will pulse the voltage at HEATER_CTRL if the command is to raise the massage stone temperature and will not pulse or bias the mosfet Q1 if the command is to cool the massage stone.

While various embodiments have been described above, it should be understood that they have been presented by way of example only, and not limitation. Thus, the breadth and scope of a preferred embodiment should not be limited by any of the above-described exemplary embodiments, but should be defined only in accordance with the following claims and their equivalents.

1. A heated massage stone, comprising:
a first ceramic portion having an outer curved surface extending to a first portion inward angled surface that extends to a first ceramic portion ledge perimeter extending away from the first portion inward angled surface at a substantially perpendicular angle;
a gasket attached to the first ceramic portion ledge perimeter, the gasket having a first gasket segment attached substantially along the inward angled surface, and a second gasket segment attached and extending along the first ceramic portion ledge perimeter,
a second ceramic portion having an outer curved surface extending to a second ceramic portion inward angled surface, the second ceramic portion inward angled surface having a second ceramic portion edge perimeter that is larger than the first ceramic portion ledge perimeter; and
a heating element coupled to a battery, the heating element and battery positioned within a cavity formed by the first ceramic portion and the second ceramic portion;
wherein the first ceramic portion and second ceramic portion are pressure fit together and sealed by the silicon gasket to enclose the heating element and battery within.

2. The heated massage stone in claim 1 wherein,
the first gasket segment is attachable to the length and width of the first ceramic portion negative edge or inward angled surface and at least a second gasket segment is attachable to the length and width of the first ceramic portion ledge perimeter.

3. The heated massage stone in claim 2 wherein,
the first gasket segment comprises first and second portions and the first portion is thicker than the second portion.

4. The heated massage stone in claim 3 wherein,
the second portion is about twice as thick as the first portion.

5. The heated massage stone in claim 2 wherein,
the second gasket segment angles or curves away from the transition from the first gasket segment to the second gasket segment at an angle of between 91 and 105 degrees relative to the first ceramic portion inward angled surface.

6. The heated massage stone in claim 2 wherein,
the second gasket segment angles away from the transition from the first gasket segment to the second gasket segment at an angle of between 1 and 15 degrees relative to a geometrical normal from the first ceramic portion inward angled surface.

7. The heating massage stone in claim 1 wherein,
the first gasket segment includes at least one protrusion extending from the second segment of the silicon gasket, the at least one protrusion selected from rectangular, curved, triangular, and cone-shaped, and extending substantially perpendicularly from the second segment.

8. The heating massage stone in claim 8 wherein,
the at least one protrusion pivots from the second segment of the silicon gasket.

9. The heating massage stone in claim 1 wherein,
the first gasket segment includes a plurality of protrusions extending from the second segment of the silicon gasket, each protrusion selected from rectangular, curved, triangular, and cone-shaped, and extending substantially perpendicularly from the second segment.

10. The heating massage stone in claim 9 wherein,
the first gasket segment includes a plurality of protrusions that pivot from the second segment of the silicon gasket.

11. A heated massage stone, comprising:
a first ceramic portion having an outer curved surface extending to a first portion inward angled surface that extends to a first ceramic portion ledge perimeter extending away from the first portion inward angled surface at a substantially perpendicular angle;
a gasket attached to the first ceramic portion ledge perimeter, the gasket having a first gasket segment attached substantially along the inward angled surface, and a second gasket segment attached and extending along the first ceramic portion ledge perimeter,
a second ceramic portion having an outer curved surface extending to a second ceramic portion inward angled surface, the second ceramic portion inward angled surface having a second ceramic portion edge perimeter that is larger than the first ceramic portion ledge perimeter; and
a heating element coupled to a battery, the heating element comprised of a substantially flat electrically conductive and resistive element embedded within a non-conductive material, and heating element and battery positioned within a cavity formed by the first ceramic portion and the second ceramic portion, the non-conductive material positioned against an inner surface of the second ceramic portion;
wherein the first ceramic portion and second ceramic portion are pressure fit together and sealed by the silicon gasket to enclose the heating element and battery within.

12. The heated massage stone in claim 11 wherein,
a charging port coupled to at least one of the first ceramic portion or second ceramic portion outer curved surfaces.

13. The heated massage stone in claim 11 wherein,
the heating element comprises a graphite foil and the non-conductive material comprises at least one silicone sheet and the heating element is embedded within the at least one silicone sheet.

14. The heated massage stone in claim 13 further comprising,
a thermistor mechanically coupled to the heating element with a cuff or sleeve fashioned in the at least one silicone sheet.

15. The heated massage stone in claim 14 further comprising,
a heater driver device coupled electrically in series with the heating element,
a microcontroller having a microcontroller output coupled to the heater driver device, the microcontroller having a microcontroller input coupled to a voltage related to the thermistor value.

16. The heated massage stone in claim 15 further comprising,
a wireless link coupled to the microcontroller that receives wireless commands from a remote control device to set the heated massage stone temperature.

17. The heated massage stone in claim 15 further comprising,
a wireless link coupled to the microcontroller that receives wireless commands from a remote control device to turn-on and turn-off the heated massage stone.
